(19) United States
(12) Patent Application Publication
(10) Pub. No.: US 2022/0378857 A1
(43) Pub. Date: Dec. 1, 2022

(54) COMPOSITION FOR THE CARDIOVASCULAR RISK REDUCTION

(71) Applicant: NEILOS S.R.L., Piano di Sorrento (IT)

(72) Inventor: Umberto DI MAIO, Piano di Sorrento (IT)

(73) Assignee: NEILOS S.R.L., Piano di Sorrento (IT)

(21) Appl. No.: 17/755,101

(22) PCT Filed: Oct. 19, 2020
(86) PCT No.: PCT/IB2020/059811

§ 371 Date: Apr. 21, 2022

(30) Foreign Application Priority Data
Oct. 21, 2019 (IT) 102019000019391

(51) Int. Cl. A61K 36/062; A61K 36/73; A61K 36/8962; A61K 9/00; A61P 9/10

(52) U.S. Cl. CPC A61K 36/062; A61K 36/73; A61K 36/8962; A61K 9/0053; A61P 9/10

(57) ABSTRACT

The present invention relates to a composition for oral use comprising at least an extract of red yeast rice, at least an extract of a plant belonging to the genus Allium and at least an extract of a plant belonging to the genus Malus and to the use thereof for the prevention and/or treatment of hypercholesterolemia, the main cardiovascular risk factor.

### FIELD OF THE INVENTION

The present invention relates to a composition for oral use comprising at least an extract of red yeast rice, at least an extract of a plant belonging to the genus Allium and at least an extract of a plant belonging to the genus Malus and to the use thereof for the prevention and/or and treatment of hypercholesterolemia, the main cardiovascular risk factor.

### STATE OF ART

Hypercholesterolemia is a pathological condition characterized by the presence of high levels of lipids (such as cholesterol, triglycerides or phospholipids) in the bloodstream. It is considered the main atherosclerotic and cardiovascular risk factor since the excessive increase in the levels of LDL cholesterol has been associated to a greater coronary risk. Therefore, the reduction in the LDL levels seems to be the best approach to be adopted for the prevention of cardiovascular disorders. The forms of familiar hypercholesterolemia (FH) are characterized by hyperproduction of cholesterol at hepatic level genetically determined and due, in most cases, to a mutation of the gene which codifies for the cell receptor of LDLs which, consequently, does not succeed anymore to bind the circulating LDL particles nor to remove them from the bloodstream. In the developed countries, dyslipidaemias very often are a consequence of the so-called metabolic syndrome and then attributable to an unhealthy lifestyle and/or eating habits. This last type of dyslipidaemia is characterized, apart from high levels of LDL and low levels of HDL, also by obesity, insulin-resistance, hypertension and diabetes of type 2. Epidemiological studies have further highlighted the involvement of other factors such as the plasmatic increase in fibrinogen and coagulation factors, the increase in the platelet activation and modifications of the oxidative state of LDLs. The drug treatment of choice for the control of dyslipidaemias consists in the administration of statins alone or in association with other active principles as inhibitors of the absorption of cholesterol or inhibitors of PCSK9, an enzyme which regulates the degradation of the LDL receptors. However, the therapy with statins has considerable limits due to the serious side effects at the muscle and hepatic level (for example, myopathies, rhabdomyolysis, alteration of hepatic functionality and increase in transaminases) and to drug-resistance and intolerance phenomena. In case of patients resistant to statins an inadequate (or lack in) reduction in the levels of LDL cholesterol occurs, accompanied by the appearance of the side effects, generally due to polymorphism of HMGCo-A reductase, of glycoprotein P, of cholesterol 7 α-hydroxylase, of apolipoprotein E, of lipoprotein A of LDL receptor whereas, in the intolerant patients, the use of statins is absolutely not recommended since the side effects appear suddenly and in the most serious form. Another factor to be considered is the fact that the treatment with statins is suitable for patients having high or very high cardiovascular risk and not for subjects having mild or moderate cardiovascular risk and they have LDL levels not far from the standard, thereto generally a simple lifestyle change is recommended. Therefore, there is still the need for solutions for the prevention and/or treatment of cholesterolaemia and, more generally, for the control of cardiovascular risk in subjects resistant or intolerant to statins or who have a low or moderate cardiovascular risk factor.

### SUMMARY OF THE INVENTION

Since many natural substances have demonstrated to be effective in the treatment of dyslipidaemias and, above all, safe, bioavailable and well tolerated by the patients, the present invention is based upon the research and identification of a combination of natural substances which, when used in association, exert an effective and potentiated action in the prevention and/or treatment of hypercholesterolemia and then which is useful for the cardiovascular risk reduction.

After extensive experimentation, the inventors have found that the association of at least an extract of red yeast rice, at least an extract of a plant belonging to the genus Allium and at least an extract of a plant belonging to the genus Malus, provides a natural solution to the clinical problem of the cardiovascular pathologies.

Therefore, the present invention relates to an association of at least an extract of red yeast rice, at least an extract of a plant belonging to the genus Allium and at least an extract of a plant belonging to the genus Malus.

The present invention also relates to compositions, preferably for oral use, comprising the association of at least an extract of red yeast rice, at least an extract of a plant belonging to the genus Allium, at least an extract of a plant belonging to the genus Malus and at least a suitable excipient and moreover a kit of portions comprising the above-mentioned active principles separated and formulated in suitable form of oral dosage for sequential or contemporary administration of the different active principles.

The present invention further relates to the use of said association, compositions and kit in the prevention and/or treatment of hypercholesterolemia, first cardiovascular risk factor, and also in the prevention and/or treatment of cardiovascular diseases such as atherosclerosis, coronary heart diseases, metabolic syndrome, stroke, predisposition to diabetes and obesity.

Thanks to the synergy of its components, the composition the present invention relates to allows to obtain at the same time: a reduction in hypercholesterolemia;
an increase in the HDL levels;
a reduction in the vascular oxidation; and
a reduction in the intestinal absorption of cholesterol.

Other advantages and features of the present invention will result evident from the following detailed description.

### Glossary

The terms used in the present description are as generally understood by the person skilled in the art, except where differently designated.

Under the term “extract”, in the context of the present description, any product tracing back to a vegetable drug is meant, including all products deriving from mechanical treatment (pulverization, grinding, mixing and/or other methods) or from extractive treatment (extraction with solvent, distillation, and/or other specific methods) performed on a drug.

### DETAILED DESCRIPTION OF THE INVENTION

As mentioned above, the present invention relates to an association comprising as main active principles at least an extract of red yeast rice, at least an extract of a plant belonging to the genus Allium and at least an extract of a plant belonging to the genus Malus.

The main active ingredients of the association of the invention are described hereinafter.

Red Yeast Rice

The red yeast rice (RYR) is a nutraceutical obtained from the fermentation of the white rice with the yeast Monascus purpureus responsible for the typical colour and the production of important secondary metabolites. The so-enriched rice includes a series of bioactive compounds, thereamong sterols, isoflavones, unsaturated fatty acids and monacolins. Among monacolins, monacolin k is the most abundant and interesting. It is also known as lovastatin since the two molecules have an almost analogous structure. In the red rice extract, monacolin k is present both in lactone form and in hydrolysed form. Generally, at low pH values the lactone form prevails which converts spontaneously into the active form of hydroxy acid at neutral or basic pH. In the metabolism of monacolin, the enzymes of the family of cytochrome P450 and glycoprotein P are involved, therefore such molecule could cause interactions with the substances or the active principles normally metabolized by means of these routes. Monacolin k represents the active responsible for the health effect which the red yeast rice has on hypercholesterolemia. Thanks to its structural features, the above-mentioned molecule, as well as the statins, is capable of inhibiting the hydroxymethylglutaryl-CoA reductase enzyme (HMGCo-A reductase) which converts hydroxymethylglutaryl-CoA into Mevalonate and which represents the limiting enzyme in the cholesterol biosynthesis process. The main side effects caused by the statins are due to the fact that the pharmacological treatment with the same causes the depletion of important metabolites such as Mevalonate, mitochondrial coenzyme Q and the regulatory proteins binding GTP, with consequences on the cellular energy metabolism, the mitochondrial function and the pathways of intracellular protein signalling. By taking into consideration this factor, the treatment with red rice results to be particularly advantageous since monacolin, even if it exerts the same action of the statins on the HMGCo-A reductase enzyme, has a markedly lower effect as far as the depletion of the cellular metabolites is concerned. Given the structural overlay existing between monacolin k and lovastatin, up to now, the use of RYR in the regulation of the plasmatic levels of cholesterol and in the prevention of the cardiovascular disorders is still very controversial, above all as to the statin-sensible patients, since there is a certain fear in incurring in the same side effects. In spite of everything, the clinical data collected in the last years reveal that, among the subjects using RYR-based supplements, the appearance of serious side effects is lower than 0.1%. Moreover, there is a huge number of clinical trials demonstrating the positive effect of monacolin in reducing the plasmatic levels of LDL and the appearance of cardiovascular events. In 2011, EFSA officially declared that there is a cause-effect relationship between the consumption of 10 mg/die of monacolin and the reduction in the level of LDL cholesterol in blood. This value was determined by considering two double-blind randomized studies, controlled with placebo, in which a reduction in the levels of total cholesterol and LDL cholesterol of 16% and 22%, respectively, is observed by administrating 7.5 mg/die of active principle for 12 weeks and a reduction in the above-mentioned outcomes of 20% and 26%, respectively, is observed by administering a dose containing 11.4 mg/die of monacolin for 8 weeks.

An in vivo study on experimental animals examined the effect of the extract of red yeast rice on the lipidic profile of the treated mice and on the vascular endothelial inflammation which is one of the phenomena leading to the establishment of the atherosclerotic process. Mice C57BL/6JApoe^{-/-} were divided into three groups:
1. Mice with normal diet, without treatment;
2. Mice with high-fat diet, without treatment;
3. Mice with high-fat diet and treatment with the extract of RYR (0.34 g/kg per day).

The mice of group 2, not treated and subjected to a high-fat diet, developed atherosclerotic plaques and high levels of total cholesterol and LDL cholesterol with respect to the group fed with a normal diet. The treatment with the extract reduced the sizes of the plaques of about 15% and the lesion area. Moreover, the results obtained in the treated group highlighted a reduction in the plasmatic levels of cholesterol with respect to the control group and to the not treated group. An analysis of the levels of protein expression on the vascular, hepatic and intestinal tissue of the sacrificed mice highlighted that:
The treatment with the red rice extract reduced the levels of expression of HMGCoA reductase at hepatic level;
RYR reduced the levels of expression, at aorta level, of TRL2, TRL4 and MAPK, pro-inflammatory proteins responsible for the activation of the cascades of signalling the inflammation and activation of some atherosclerotic genes;
The extract increased the levels of expression of JAM-1 and of occludins at intestinal level, by demonstrating to improve the intestinal functions of the mice Apoe^{-/-} and, then, to reduce the inflammation risk.

The effectiveness of the extract of red yeast rice from Monascus purpureus was tested in a great randomized, multicentric, double-blind study, controlled with placebo, performed on 4870 Chinese patients between 18 and 70 years old with a preceding documented myocardial infarction, with high levels of creatin kinase and with a medium level of LDL equal to 129 nng/dL.

The patients were divided into two groups:
Treated: 600 mg per day of xuezhikang, the extract of the Chinese red rice (each capsule includes 300 mg of extract corresponding to 2.5-3.2 mg of monacolin);
Placebo

The subjects were kept under control for a period of about 4 years and a half and it was evaluated: the appearance of coronary events, the mortality for cardiovascular problems and the levels of plasmatic lipids. The results extrapolated from this study showed that in the subjects of the treated group there was a reduction in the levels of LDL cholesterol of about 20% with respect to placebo. The total lipidic profile of the patients results to be improved, with a reduction in the levels of triglycerides and an increase in HDL cholesterol. A reduction in the incidence of cardiovascular events and, more generally, of the mortality attributable to cardiovascular problems was noticed. The study did not notice any case of serious side effects.

A meta-analysis grouped 20 randomized trials with 6653 participants. The considered studies compared the red rice extract containing monacolin k (with doses comprised between 4.8 and 24 mg per day) with placebo or with statins. The results showed that the supplementation with RYR reduces the levels of LDL with respect to placebo. When the treatment was compared to statins, no difference was noted among the groups, thus by demonstrating that monacolin k has an effectiveness comparable to the traditional treatment.

Garlic

Garlic, Allium sativum L., is a bulbous cultivated plant, traditionally assigned to the family of Liliaceae, but which the recent APG III classification attributes to Amaryllidaceae. This plant holds a position of great importance both in the field of nutrition and in the popular medicine in many cultures all over the world. It is widely used as seasoning, but relevant healthy properties have always been attributed to it. In fact, in the folklore of many cultures, garlic is considered a formidable prophylactic and therapeutic agent. In the ancient Indian and Chinese medicine, it was considered an agent useful to improve digestion and to help respiration, as well as a therapeutic agent in the treatment of leprosy and parasitic disorders. In the Egyptian medicine treaties garlic was considered useful in the treatment of the cardiac disorders, in tumours, in helminthiases and in other disorders. Many properties, which were attributed to this plant in past, nowadays are still handed down very much in the common knowledge. Garlic seems to play an important role in the treatment of infections, of gynaecological disorders, of toothache and of arthritis, in the prevention of cancer, in the pathogenesis of the cardiovascular disorders and in the chronic diseases linked to age. In the last years the beneficial properties which garlic has on the cardiac well-being and on the cardiovascular risk reduction have been attracting much interest.

The results of chemical analyses designate that about 65% of fresh garlic consists of water, the remaining portion consists of carbohydrates, mainly polymers of fructose called fructans, organosulfur compounds, proteins, fibres and free aminoacids. It further includes high levels of saponins, phosphorus, potassium, sulphur, zinc and low levels of selenium, vitamin A and C, calcium, magnesium, sodium, iron, manganese and vitamins of group B. The main organosulfur compounds present in garlic are γ-glutamyl-S-allyl-L-cysteine (G-SAC) which, during storage or when they are subjected to reactions such as Maillard reactions, are hydrolysed and oxidized to S-allyl cysteine (SAC), or alliin and to S-allylmercaptocysteine (SAMC). Such molecules are considered to be those with greater antioxidant power, as well as the real responsible for the beneficial effect at the cardiovascular level. Some studies show that the water-soluble compounds present in garlic are the most effective in reducing the levels of cholesterol. Thereamong, alliin and, to a much greater extent, allicin which forms therefrom thanks to the action of the alliinase enzymes. The protective mechanism responsible for such beneficial effect is attributed to several action mechanisms. In several in vitro studies the tested garlic extracts demonstrated to be capable of inhibiting the enzymes responsible for the biosynthesis of cholesterol and of the fatty acids such as squalene monooxygenase and HMGCoA reductase. Moreover, garlic is active in reducing the oxidative stress which represents one of the etiological factors of pathogenesis of the cardiovascular disorders, in reducing the oxidation of LDLs, in reducing total triglycerides and in increasing HDLs.

An in vivo study performed on experimental animals examined the effect that the garlic extract can have on a metabolic and vascular profile altered due to an obesity condition. To this purpose, the mice subjected to the test were divided into two groups:
12 mice fed with a standard diet;
24 mice subjected to a high-fat diet.

Half of the considered mice were treated with the garlic extract (250 mg/kg) and the other half only with a carrier (5 mL/kg) for a month, before being sacrificed. On the collected serum samples the levels of triglycerides, total, LDL and HDL cholesterol were measured. The obtained results demonstrated that the mice subjected to a high-fat diet developed a hyperlipidaemia characterized by high levels of triglycerides, cholesterol and LDL and low levels of HDL with respect to control.

The treatment with garlic, even if it does not modify the concentration of total cholesterol, reduces the levels of LDL and increases the HDL ones. On the perivascular adipose tissue collected soon after the sacrifice, a RT-PCR was performed to evaluate the levels of gene expression of some pro-inflammatory proteins. The results showed that the treatment reduces the levels of expression of the pro-inflammatory markers IL-1β, TNF-α and iNOS by reducing the risk of inflammation of the vascular epithelium and, then, by improving the functionality of the whole tissue.

The hypocholesterolaemic action of garlic was investigated in a randomized, double-blind, clinical study, controlled with placebo, performed on 42 men who were between the age of 35 and 70 with a moderate hypercholesterolemia. The subjects selected for the study were assigned to two distinct groups:
1. Treated: 600 mg of dry extract per day;
2. Control: with placebo.

The levels of total, LDL and HDL cholesterol were measured after 8 weeks and after 12 weeks of treatment. At the end of the study, the levels of total cholesterol of the treated group reduced by 7.6% with respect to the beginning of treatment and by 11.5% with respect to the group which received placebo. The measurements made after 8 and 12 weeks of study highlighted that the LDL cholesterol of subjects in the treated group reduced by 11.8% and by 13.8%, respectively. The treatment with garlic further highlighted an increase in the levels of HDL cholesterol which, at the end of the study, increased by 11.5%.

The reported data then show that garlic has moderate but significative effect in reducing cholesterol and constitutes a valid alternative to the pharmacological treatments for reducing hypercholesterolemia in view of a cardiovascular risk reduction.

Apple

Malus pumila var. Annurca is the only apple variety native to Southern Italy, mainly in the region Campania between Caserta and Benevento. Annurca apple has a pulp rich in juice, a compact, crunchy pulp and characterized by the typical sour taste which distinguished it from other apple varieties. Healthy properties have always been attributed to apple. Nowadays it is known that the benefits brought by the consumption of apples can be attributed to the polyphenolic content of the fruit constituted by quercetin, epicatechin, catechin, procyanidins, anthocyanins, dihydrochalcones and phenolic acids. Even if the components are substantially the same, the specific activities vary between the different apple varieties. What makes annurca apple so interesting and promising from the nutraceutical point of view indeed is its very high wholly characteristic polyphenolic content. Generally, the polyphenols can be present under form of dimers, trimers, tetramers, pentamers and oligomers having high molecular weight. In Annurca apple the polyphenolic fraction is above all rich in procyanidins, in particular procyanidin B2, and oligomers formed by a number of monomers comprised between 6 and 8. The apple extract is known having chemoprotective properties. Several studies, in fact, highlighted that the fresh apple extract is capable of inhibiting the cellular growth in various types of cancer, like colon and liver cancer, in dose-dependent way. Moreover, several products based upon apple polyphenolic extracts are on the market, proposed for the body weight control, such as cosmetics, for the anti-inflammatory action. Recent studies highlight that some polyphenols contained in the apple have beneficial effect in the patients affected by metabolic syndrome. Phlorizin is the main responsible for the hypoglycaemic action. In vitro and in vivo studies demonstrate the hypoglycaemic effect by highlighting that the compound in question induces glucosuria and blocking of intestinal absorption of glucose.

The hypolipidemic effect can be mainly attributed to procyanidins and this makes the polyphenolic extract of Annurca very promising in this field. The complex action mechanism of polyphenols of Annurca apple was exposed by several in-vitro studies. The beneficial effect on hypercholesterolemia is due to the combined action of dimeric procyanidins (in particular procyanidin B2) and of oligomers. Both these fractions of Annurca polyphenolic complex are responsible for the hypolipidemic effect, but with different action mechanisms. The several performed pre-clinical studies, in particular the studies on bioavailability and bioaccessibility of the polyphenols of Annurca apple confirm the hypothesis according thereto the oligomers having high molecular weight are not absorbed at intestinal level. Up to now it has been demonstrated that oligomers accumulate in the intestine and act here locally to reduce the levels of cholesterol by acting in a way similar to β-cyclodextrins and forming complexes similar to micelles incorporating cholesterol and preventing the absorption thereof. On the contrary, dimers are quickly absorbed in the intestinal tract and reach quickly liver wherein, indirectly, they are involved in the increase in the LDL receptors. Therefore, the dimeric procyanidins are capable of reducing the levels of LDL cholesterol with the same effectiveness of statins and, differently from these latter which have no effect on HDL, form stable complexes with apolipoprotein A1, main protein structural component of HDL, and increase the inverted route of cholesterol and, consequently, the levels of eliminated cholesterol.

A preliminary clinical study performed on 250 patients with a moderate hypercholesterolemia evaluated the effect that the ingestion of two Annurca apples per day exerts on the lipidic profile of the considered subjects compared to that of other apple varieties. The results of the study demonstrated that, with respect to other highlighted apple varieties, Annurca apple is responsible for a significative reduction in the levels of total cholesterol and LDL cholesterol and the increase in the levels of HDL.

Based upon these data a monocentric, double-blind, clinical study was prepared, controlled with placebo, lasting 16 weeks on 250 subjects with mild and moderate hyperlipidaemia. The subjects selected for the study (116 men and 134 women) were aged between 30 and 83 and had the following basal levels of cholesterol: total cholesterol 214-254 mg/dL; LDL cholesterol 150-205 mg/dL; HDL cholesterol 30-43 mg/dL. The participants in the study were subjected to a first study phase of 4 weeks, in which a placebo was administered, followed by phase two of 8 weeks, wherein two capsules per day of polyphenolic extract of Annurca apple containing each one 400 mg were administered, to finish with 4 weeks of follow-up. In the placebo phase variations in the levels of total, LDL and HDL cholesterol were not recorded. The results recorded in phase 2 showed, already after one month of treatment, a reduction in the levels of total and LDL cholesterol of 24.9% and of 37.5% respectively, and HDL increase of 49.2%.

The reported data then show that the polyphenolic extract of Annurca apple constitutes a valid alternative to the therapeutic treatment for controlling hypercholesterolemia in subjects with moderate alterations of the lipidic profile. Moreover, the use of nutraceutical products based upon Annurca apple is safe and no type of side effect is recorded.

The present invention relates to an association of at least an extract of red yeast rice, at least an extract of a plant belonging to the genus Allium and at least an extract of a plant belonging to the genus Malus.

Under the wording “at least an extract of red yeast rice” in the context of the present invention it is meant that the composition can include at least one, two, three or more extracts of red yeast rice. Under “red rice extract” the rice yeast product inoculated with the red yeast, Monascus purpureus is designated. According to an embodiment, the red yeast rice can be any extract preferably titrated at a certain percentage of monacolin k.

To the purpose of the present invention, extracts of red yeast rice having a titre in total monacolin K (acid form plus lactone form) of at least 0.1%, preferably of about 0.3 to about 10% can be used. For example, an extract of red yeast rice suitable to the implementation of the present invention can have a titre in monacolin k from 0.5% to 5%.

In particular, the authors of the invention showed that amounts comprised between 1 and 10000 mg per day of extract of red yeast rice in combination to at least an extract of a plant belonging to the genus Allium and at least an extract of a plant belonging to the genus Malus as herein described are effective, by obtaining the reduction in plasmatic cholesterol in the treated subjects.

By pure way of example, but in no way with limitative purposes, 1 capsule/die comprising 333.3 mg of extract of red yeast rice titrated at 3% in monacolin K, corresponding to about 10 mg of monacolin K per capsule, for a total of 10 mg/die, can be administered.

Under the wording “at least an extract of a plant belonging to the genus Allium” in the context of the present description it is meant that the composition can include at least one, two, three or more extracts selected among Allium sativum, Allium ursinum and Allium cepa, preferably Allium sativum. The extract could be prepared according to the procedures known in the state of art, for example it could be a dry extract of the bulb or an essential oil of Allium sativum titrated at 1% in allin. The garlic extract, for example, will be prepared by means of extraction in a mixture of ethanol/water. The garlic extract for example could be administered with a dosage regime comprised between 1 and 10000 mg/die, in particular between 10 and 2000 mg/die.

Under the wording “at least an extract of a plant belonging to the genus Malus” in the context of the present description it is meant that the composition can comprise at least one, two or more extracts selected among Malus pumila or Malus domestica var. Annurca, preferably Malus pumila cultivar Annurca. The extract could be prepared according to the procedures known in the state of art, for example it could be a dry extract of Malus pumila having high content in polyphenols. The extract, for example, could be prepared by means of extraction in water of the fruit and optionally lyophilized. The apple extract, in particular di Malus pumila var. annurca, could be administered for example with a dosage regime comprised between 1 and 10000 mg/die, in particular between 10 and 3000 mg/die, preferably in 1-2 separate administrations (for example 400 mg×2 times per day, or 400 mg 1 time per day).

The person skilled in the art could adapt the amount of extract used in the preparation of the formulations to be administered according to the used extract of red yeast rice, Allium and Malus. The general practitioner will be able to identify the optimum dosage for the subject to be treated based upon age, sex, weight and general health state. Therefore, the dosage of the single active principles can be adapted, even during the period of assumption of the association or composition of the invention according to the results obtained over time.

The inventors have detected the optimal pro dose (or per single dosage unit) dosages of each extract.

The pro dose dosage of extract of red yeast rice is comprised between 1 mg and 10000 mg, preferably between 10 mg and 3000 mg, still more preferably between 20 mg and 2000 mg. The pro dose dosage of extract of plant belonging to the genus Allium is comprised between 1 mg and 10000 mg, preferably between 10 mg and 2000 mg, still more preferably between 20 mg and 1000 mg. The pro dose dosage of extract of plant belonging to the genus Malus is comprised between 1 mg and 10000 mg, preferably between 10 mg and 3000 mg, still more preferably between 20 mg and 2000 mg.

The inventors have observed that there is the most synergy between the different active principles when:
the extract of red yeast rice is present in an amount comprised between 1 mg and 10000 mg, preferably between 10 mg and 3000 mg, still more preferably between 20 mg and 2000 mg per dosage unit;
the extract of plant belonging to the genus Allium is present in an amount comprised between 1 mg and 10000 mg, preferably between 10 mg and 2000 mg, still more preferably between 20 mg and 1000 mg per dosage unit; and
the extract of plant belonging to the genus Malus is present in an amount comprised between 1 mg and 10000 mg, preferably between 10 mg and 3000 mg, still more preferably between 20 mg and 2000 mg per dosage unit.

The association of the present invention could include one or more extracts of each one of the herein described active principles, provided that it comprises at least an extract of red yeast rice, at least an extract of a plant belonging to the genus Allium and at least an extract of a plant belonging to the genus Malus.

According to another embodiment the association comprises extract of red yeast rice, extract of Allium sativum and extract of Malus pumila var. annurca.

The present invention further relates to compositions comprising the association according to any one of the herein described embodiments and to one or more suitable excipients.

According to an embodiment, the composition comprises, as main active ingredients, extract of red yeast rice, extract of Allium sativum and extract of Malus pumila var. annurca and one or more excipients as defined hereinafter.

In an additional embodiment, the composition comprises at least an extract of red yeast rice, at least an extract of a plant belonging to the genus Allium and at least an extract of a plant belonging to the genus Malus as single active principles in reducing hematic cholesterol and/or cardiovascular risk.

Therefore, the composition can comprise other components, such as for example excipients, carriers, stabilizers, preserving agents and the like and/or other active principles, provided that said other active principles are not indicated in the reduction in the plasmatic cholesterol and/or cardiovascular risk in general.

The compositions according to any one of the embodiments provided in the present description, can be formulated in any form and by any administration route and associated to any other component, in a variety of ways.

According to an embodiment, the compositions of the invention are compositions for oral use in solid form such as, for example, powder, orodispersible powder, granulate, hard capsule or soft-gel, tablet, sachet, pill or gelatine or in liquid form such as, for example, emulsions, solutions, prepared or extemporaneous suspensions, syrups and elixirs.

Suitable excipients can be selected among those usually known in the state of art and include, but they are not limited thereto: diluents (for example dibasic calcium phosphate, lactose, microcrystalline cellulose and cellulose derivatives), thickeners (for example gums, hydroxypropyl methylcellulose and other cellulose derivatives), sweeteners (for example sorbitols, mannitol and other polyols, acesulfame K, aspartame, cyclamates, saccharin, sucralose), lubricants (for example magnesium stearate, stearic acid, waxes), dispersants, surfactants (for example sodium lauryl sulphate and polysorbates), flavouring agents, adsorbents (for example silica gel, talcum, starch, bentonite, kaolin), glidants and anti-adhering agents (for example talcum, colloidal silica, corn starch, silicon dioxide), dyes (for example iron oxides), opacifiers (for example titanium oxide), anti-oxidants, binders (for example gums, starch, gelatine, cellulose derivatives, sucrose, alginate sodium), disaggregating agents (starch, microcrystalline cellulose, alginic acid, crospovidone), plasticizers (for example ethylcellulose and other cellulose derivatives, acrylates and matacrylates, glycerol and sorbitol), preserving agents (for example parabens, sulphur dioxide), viscosifiers, emulsifiers, humectants, wetting agents, chelating agents and mixtures thereof.

The above-mentioned solid dosage forms could be coated with enteric, gastric coatings or coatings of other type known in the state of art.

They can allow the release of the active ingredients only or preferably in a certain tract of the intestine, in case, in delayed way. Substance which can allow such delayed use include, but they are not limited thereto, polymers and waxes.

The compositions of the present invention, for example, will be a medical device, food supplement, a nutraceutical, dietetic or nutritional composition, foodstuff, a beverage, food, a nutraceutical, medicated food, food for special medical purposes, or a pharmaceutical composition.

The compositions of the present invention could further be, for example, simple feed, complementary feed, complete feed, feed intended to particular nutritional purposes or medicated feed.

The compositions according to any one of the herein described embodiments could be used both by human beings and by animals.

The combination of the above-mentioned active principles could be used formulated in one single composition according to the various above-described embodiments or in a kit comprising the different separate ingredients, for example, in compositions formulated in suitable oral dosage form as defined above for the sequential or contemporary administration of the different active principles.

Therefore, the present invention also relates to a kit of portions including the different active principles of the association according to any one of the herein described embodiments separated and formulated in suitable oral dosage form for sequential or contemporary administration of the different active principles.

As it will result evident to the person skilled in the art from the herebelow provided examples, the present invention constitutes a valid support for the reduction in hypercholesterolemia, main cardiovascular risk factor, thanks to the combined and diversified action of its components. Without wanting to be linked to any specific theory, the inventors came to the conclusion that the red yeast rice, rich in monacolin k, contributes to reduce the plasmatic levels of cholesterol by inhibiting the biosynthesis thereof at hepatic level and, at the same time, by reducing the phenomena of vascular oxidation; the organosulfur compounds present in the garlic extract potentiate the action of monacolin both at hepatic level and at vascular level and they contribute to increase the levels of HDLs; procyanidins of Annurca apple reduce the plasmatic levels of cholesterol by increasing the receptors of LDLs and by reducing the intestinal absorption thereof and, at the same time, they increase the levels of HDL. The action of the single components and of the association the present invention relates to was evaluated by meant of in vitro and/or in vivo test which, besides, highlighted the way in which the composition of the invention is particularly effective thanks to the action of (synergically) reinforcement of its components.

Therefore, the present invention further relates to the association, to the compositions and to the kit, according to any one of the herein described embodiments, for use in the treatment and/or in the prevention of hypercholesterolemia, both in humans and in animals.

Moreover, the present invention also relates to the association, to the compositions and to the kit, according to any one of the herein described embodiments, for use in the prevention and/or in the treatment of disorders affecting the cardiovascular apparatus such as, for example, atherosclerosis, coronary heart diseases, metabolic syndrome, stroke, predisposition to diabetes and obesity, both in humans and in animals.

The association of the above-mentioned active principles could be formulated in one single composition or in a kit according to the various above-described embodiments and prepared for example by mixing the selected active principles with possible other excipients as it is known to the person skilled in the art.

### EXAMPLES

Some not limiting examples of the compositions according to the present invention are reported herebelow. In the examples the percentages are to be meant as weight percentages.

### Example 1: Tablet

| | Amount present for single |
|---|---|
| Active principle | dosage unit |
| Extract of red yeast rice of | 10 mg |
| which monacolin K | |
| Annurca apple | 800 mg |
| Allium sativum e.s. | 100 mg |

### Example 2: Capsule

| | Amount present for single |
|---|---|
| Active principle | dosage unit |
| Extract of red yeast rice of | 8 mg |
| which monacolin K | |
| Annurca apple | 500 mg |
| Allium sativum e.s. | 300 mg |

### Example 3: Coated Tablet

| | Amount present for single |
|---|---|
| Active principle | dosage unit |
| Extract of red yeast rice of | 3 mg |
| which monacolin K | |
| Annurca apple | 400 mg |
| Allium sativum e.s. | 300 mg |

### Example 4: Tablet

| | Amount present for single |
|---|---|
| Active principle | dosage unit |
| Extract of red yeast rice of | 10 mg |
| which monacolin K | |
| Annurca apple | 400 mg |
| Allium sativum e.s. | 250 mg |

### Example 5: Tablet

| | Amount present for single |
|---|---|
| Active principle | dosage unit |
| Extract of red yeast rice of | 2.5 mg |
| which monacolin K | |
| Annurca apple | 400 mg |
| Allium sativum e.s. | 50 mg |

### Experimental Evidence

The authors of the present invention have observed that the administration by oral route of a composition comprising the association of at least an extract of red yeast rice, at least an extract of a plant belonging to the genus Allium, in particular Allium sativum, and at least an extract of a plant belonging to the genus Malus, in particular, Malus pumila var. annurca results to be effective in the reduction in the plasmatic cholesterol and in the cardiovascular risk in general, also thanks to the synergic action of its components.

In order to evaluate the anti-inflammatory action of the formulation in question, in vitro tests on murine cells or of human derivation can result to be useful. The cells have grown in complete DMEM growth medium and kept for example at 37° C. and in humid atmosphere with 5% of CO₂. After treatment with the composition of the invention, the total RNA extracted from the samples is subjected to specific treatments such as for example RT-PCR to evaluate the levels of expression of pro-inflammatory proteins such as for example IL-1β, TNF-α and iNOS.

An in-vitro test to evaluate the levels of oxidative stress consists in treating the cells with specific agents such as for example 2,7-dichlorodihydrofluorescein acetate. The generated fluorescence can be measured by means of fluorescence microscope at 529 nm. Other methods conventionally known to the person skilled in the art can be used to evaluate the antioxidant activity of the formulation of the present invention. For example, it is possible to study the expression of SOD1 and NOX4 under conditions of stimulus induced by LPS or hyperglycaemia before and after treatment with the substances the present invention relates to. In order to evaluate the uptake of cholesterol an in-vitro test can be performed on a murine or human cell line. The treated and not treated cells are incubated with marked cholesterol and, subsequently, washed to remove the excess. At the end of the test the radioactivity of the solubilized cells is measured. By using the latter test, one could appreciate that the composition of the present invention can involve a reduction in the uptake of cholesterol in a range from 1 to 80% with respect to the single ingredients. The effectiveness of the present invention can be evaluated even by means of an in vivo test on experimental animals in line with the directives of the European Community and of the Ministry of Health and approved by an Ethics Committee. The test is performed on mice Apoe which, after a period of acclimatation, are divided into groups and subjected to the treatment. At time zero and at established intervals, until the end of the treatment period, plasma samples are collected and the levels of total cholesterol, LDL cholesterol and HDL cholesterol are measured.

At the end of the treatment period ex-vivo tests can be performed. Therefore, the mice are sacrificed and samples of hepatic, intestinal and arterial tissue are collected. The collected and lysed tissues are subjected to a western blot to evaluate the levels of some proteins of interest in particular, on the hepatic tissue the levels of HMG-CoA reductase and squalene monoxidase can be evaluated, on the arterial tissue the levels of pro-inflammatory proteins such as IL-1β, TNF-α, TLR2, TLR4 and MAP-kinase can be evaluated and on the intestinal tissue the total functionality of the tissue of interest can be evaluated by evaluating the levels of junction proteins such as occludins or JAM-1.

In order to evaluate the synergic activity of the composition of the present invention any one of the herein described in-vitro or in vivo tests can be used. In particular, by using the above-mentioned tests it was possible to verify that the association of at least an extract of red yeast rice with at least an extract of a plant belonging to the genus Allium and with at least an extract of a plant belonging to the genus Malus allows a reduction in the intestinal absorption of cholesterol/of plasmatic cholesterol significantly higher with respect to what is obtained with the administration of the single active principles or of only two active principles.

1. An association of at least an extract of red yeast rice, at least an extract of a plant belonging to the genus Allium and at least an extract of a plant belonging to the genus Malus.

2. The association according to claim 1, wherein the extract of plant belonging to the genus Allium is selected among Allium sativum, Allium ursinum and Allium cepa and preferably is Allium sativum and the extract of plant belonging to the genus Malta is selected among Malta pumila or Malus domestica var. Annurca and preferably is Malus pumila var. Annurca.

3. A composition comprising the association of claim 1 and one or more excipients.

4. The composition according to claim 3 wherein:
the extract of red yeast rice is present in an amount comprised between 1 mg and 10000 mg; and/or
the extract of a plant belonging to the genus Allium is present in an amount comprised between 1 mg and 10000 mg; and/or
the extract of a plant belonging to the genus Malta is present in an amount comprised between 1 mg and 10000 mg.

5. An oral composition comprising the composition of claim 3.

6. The oral composition according to claim 5 in a form selected from powder, orodispersible powder, granulate, hard capsule, soft-gel capsule, tablet and sachet.

7. The composition according to claim 3 which is in the form of a medical device, food supplement, nutraceutical, dietetic or nutritional composition, foodstuff, beverage, food, medicated food, food for special medical purposes, or pharmaceutical composition or even in the form of simple, complementary, complete feed, intended for particular nutritional purposes or medicated feed.

8. A method of preventing or treating hypercholesterolemia in a subject, comprising administering a therapeutically effective amount of the composition of claim 3 to the subject.

9. A method of preventing or treating cardiovascular disease in a subject, comprising administering a therapeutically effective amount of the composition of claim 3 to the subject.

10. The method of claim 9, wherein the cardiovascular disease is atherosclerosis, coronary heart disease, metabolic syndrome, stroke, or predisposition to diabetes and obesity.

11. The composition according to claim 4, comprising:
between 10 mg and 3000 mg of the extract of red yeast rice; and/or
between 10 mg and 2000 mg of the extract of a plant belonging to the genus Allium; and/or
between 10 mg and 3000 mg of the extract of a plant belonging to the genus Malts.

12. The composition according to claim 4, comprising:
between 20 mg and 2000 mg of the extract of red yeast rice; and/or
between 20 mg and 1000 mg of the extract of a plant belonging to the genus Allium; and/or
between 20 mg and 2000 mg of the extract of a plant belonging to the genus Malts.

13. The method according to claim 8, wherein the subject is a human.

14. The method according to claim 8, wherein the subject is an animal.
